(19) United States
(12) Patent Application Publication
(10) Pub. No.: US 2026/0151032 A1
(43) Pub. Date: Jun. 4, 2026

(54) SYSTEMS AND METHODS FOR OBTAINING MEASUREMENTS USING A COMPUTING DEVICE

(71) Applicant: Warby Parker Inc., New York, NY (US)

(72) Inventors: Dee Celeste Goldberg, New York, NY (US); David J. DeShazer, New York, NY (US); Corey Frederick, New York, NY (US); Jordan Tumlin, New York, NY (US); Brian Bernberg, New York, NY (US)

(21) Appl. No.: 18/290,646

(22) PCT Filed: Nov. 17, 2023
(86) PCT No.: PCT/US2023/080299

§ 371 Date: Jan. 19, 2024

Related U.S. Application Data
(60) Provisional application No. 63/426,929, filed on Nov. 21, 2022.

(51) Int. Cl. A61B 3/11; G06T 7/50; G06T 7/60

(52) U.S. Cl. CPC A61B 3/111; G06T 7/50; G06T 7/60; G06T 2207/30201

(57) ABSTRACT

A computing device may include a non-transitory machine readable storage medium and at least one processor configured to obtain an image including a first object and a second object; localize the second object in the image; determine a scaling factor based on at least one of the first object in the image and the second object in the image; convert coordinates of a first landmark associated with the first object in the image and a second landmark associated with the second object in the image from a first unit of measure to a second unit of measure based on the scaling factor; and calculate and store at least one measurement between the first landmark associated with the first object in the image and the second landmark associated with the second object in the image. Methods and non-transitory machine readable storage media also are disclosed.

### CROSS-REFERENCE TO RELATED APPLICATIONS

This application claims priority to U.S. Provisional Patent Application No. 63/426,929, filed Nov. 21, 2022, the entirety of which is incorporated by reference herein. This application also incorporates by reference the entireties of U.S. Pat. No. 10,825,260, entitled “Virtual Try-On Spectacle Placement Algorithm”; U.S. Patent Application Publication No. 2021/0393121, entitled “System and Method for Measuring Pupillary Distance and Uses Thereof”; and U.S. patent application Ser. No. 17/856,482, entitled “Systems and Methods for Spectacle Removal and Virtual Try-On.” All patents, patent applications, publications and products mentioned herein are hereby incorporated by reference in their entireties.

### FIELD

The present disclosure generally relates to the technical field of opticianry. More specifically, the disclosed systems and methods relate to obtaining various measurements for use in opticianry using a computing device.

### BACKGROUND

Pupillary distance (“PD”) is the distance between pupils of a subject, such as a human. The accuracy of this distance is a determining factor in the comfort and fit of eyeglasses, sunglasses, or virtual reality headsets. Mis-measured PD can lead to lenses with prismatic effects that cause headaches, blurry vision, eyestrain, nausea, vertigo, dizziness, disorientation and other issues to the subject. Traditionally, PD measurement is absent from eyeglasses or sunglasses prescriptions, but it is required for fulfilling custom prescription eyeglass or sunglass orders. The average PD for an adult human is about 63 mm. However, the PD measurement can vary widely between 51 mm and 74.5 mm for women and 53 mm and 77 mm for men. PD measurements typically are obtained when fitting eyeglasses or sunglasses with progressive lenses because a precise lens-to-pupil alignment helps to ensure comfortable vision at all distances.

Currently, there are various ways to measure PD. For example, there are two primary methods for PD measurement: single PD and dual PD. Single PD (or binocular PD) is the measurement from pupil to pupil between each eye. Dual PD (or monocular PD) is the measurement from the nose bridge to each eye.

There is a need to develop improved systems and methods for measuring PD that increase accuracy, efficiency, reliability, convenience, and use, while decreasing or eliminating human intervention and/or human error.

### SUMMARY

In some embodiments, a method may include obtaining an image that includes a first object and a second object. The second object in the image may be localized using at least one processor. A scaling factor may be determined based on at least one of the first object in the image and the second object in the image using the at least one processor. Coordinates of a first landmark associated with the first object in the image and a second landmark associated with the second object in the image may be converted from a first unit of measure to a second unit of measure based on the scaling factor using the at least one processor. At least one measurement between the first landmark associated with the first object in the image and the second landmark associated with the second object in the image may be calculated using the at least one processor, and the at least one measurement may be stored in a non-transitory machine readable storage medium that is disposed in communication with the at least one processor.

In some embodiments, the image may include two-dimensional image data.

In some embodiments, determining the scaling factor may include determining, using the at least one processor, a first dimension of the first object in the image. The first dimension of the first object in the image may be in a first unit of measure. The first dimension of the first object may be divided by a value in the second unit of measure.

In some embodiments, a method may include obtaining the value in the second unit of measure from a model of the first object.

In some embodiments, the value of the second unit of measure may be a predetermined constant value.

In some embodiments, the first object may include frames for spectacles, and the value may correspond to a height of a lens for the frames.

In some embodiments, determining the first dimension may include determining a distance between the first landmark associated with the frames for spectacles in the image and a third landmark associated with the frames for spectacles in the image.

In some embodiments, determining the scaling factor may include determining a distance from a camera to the second object and dividing the distance from the camera to the second object by a focal length of the camera.

In some embodiments, the second object may include a pupil.

In some embodiments, the image may be obtained from a camera in communication with the at least one processor. A method may include segmenting the first object in the image using the at least one processor.

In some embodiments, the image may be a composite image based on a first image obtained from a camera and a model of the first object superimposed on the first image.

In some embodiments, a non-transitory machine readable storage medium may have machine-executable instructions stored therein. When the instructions are executed by one or more processors, the one or more processors may perform a method. The method may include obtaining an image including a first object and a second object, localizing the second object in the image using the at least one processor, and determining a scaling factor based on at least one of the first object in the image and the second object in the image using the at least one processor. Coordinates of a first landmark associated with the first object in the image and a second landmark associated with the second object in the image may be converted from a first unit of measure to a second unit of measure based on the scaling factor using the at least one processor, and at least one measurement between the first landmark associated with the first object in the image and the second landmark associated with the second object in the image may be calculated using the at least one processor. The at least one measurement may be stored in a non-transitory machine readable storage medium that is disposed in communication with the at least one processor.

In some embodiments, the image may include two-dimensional image data.

In some embodiments, determining the scaling factor may include determining a first dimension of the first object in the image. The first dimension of the first object in the image may be in a first unit of measure. The first dimension of the first object may be divided by a value in the second unit of measure.

In some embodiments, a method may include obtaining the value in the second unit of measure from a model of the second object.

In some embodiments, the value of the second unit of measure may be a predetermined constant value.

In some embodiments, the first object may include a frame for spectacles. The value may correspond to a height of a lens for the frame.

In some embodiments, determining the first dimension may include determining a distance between the first landmark associated with the frame for spectacles in the image and a third landmark associated with the frame for spectacles in the image.

In some embodiments, the first object may include a frame for spectacles. The first landmark may correspond to a first midpoint of a first portion of the frame configured to at least partially surround a first lens. The third landmark may correspond to a second midpoint of the first portion of the frame configured to at least partially surround the first lens. The first midpoint may be disposed on an opposite side of an opening sized and configured to receive the first lens as the second midpoint.

In some embodiments, the first object may include a frame for spectacles. The first landmark may correspond to a first midpoint of a first portion of the frame configured to at least partially surround a first lens. The second landmark may correspond to a second midpoint of a second portion of the frame configured to at least partially surround a second lens.

In some embodiments, at least one of the first landmark and the third landmark may be identified using a processor.

In some embodiments, at least one the first landmark and the second landmark may be identified using a processor in response to receiving user input.

In some embodiments, the processor is configured to cause a graphical user interface to be displayed on a display. The graphical user interface is configured to receive input from a user for identifying at least one of the first landmark and the third landmark.

In some embodiments, the processor is configured to cause a graphical user interface to be displayed on a display. The graphical user interface is configured to receive input from a user for adjusting a location of at least one of the first landmark and the third landmark.

In some embodiments, the processor is configured to cause a graphical user interface to be displayed on a display. The graphical user interface is configured to receive input from a user for identifying the first landmark and the third landmark.

In some embodiments, the processor is configured to cause a graphical user interface to be displayed on a display. The graphical user interface is configured to receive input from a user for adjusting a location of the first landmark and the third landmark.

In some embodiments, determining the scaling factor may include determining a distance from a camera to the second object and dividing the distance from the camera to the second object by a focal length of the camera.

In some embodiments, the second object may include a pupil.

In some embodiments, the image may be obtained from a camera in communication with the at least one processor, and a method may include segmenting the first object in the image.

In some embodiments, the image may be a composite image based on a first image obtained from a camera and a model of the first object superimposed on the first image.

In some embodiments, a computing device may include a non-transitory machine-readable storage medium and at least one processor in communication with the non-transitory machine-readable storage medium. The at least one processor may be configured to obtain an image including a first object and a second object, localizing the second object in the image, determine a scaling factor based on at least one of the first object in the image and the second object in the image, and convert coordinates of a first landmark associated with the first object in the image and a second landmark associated with the second object in the image from a first unit of measure to a second unit of measure based on the scaling factor. The at least one processor may be configured to calculate at least one measurement between the first landmark associated with the first object in the image and the second landmark associated with the second object in the image and to store the at least one measurement in the non-transitory machine readable storage medium.

In some embodiments, the image may include two-dimensional image data.

In some embodiments, the at least one processor may be configured to determine a first dimension in a first unit of measure of the first object in the image and to divide the first dimension of the first object by a value in the second unit of measure to determine the scaling factor.

In some embodiments the at least one processor may be configured to obtain the value in the second unit of measure from a model of the first object.

In some embodiments, the model of the first object may be stored in the non-transitory machine-readable storage medium.

In some embodiments, the model of the first object may be stored in a second non-transitory medium that is remote from the computing device and may be accessible via a network.

In some embodiments, the value of the second unit of measure may be received from a user via an input device of the computing device.

In some embodiments, the first object may include frames for spectacles. The value may correspond to a height of a lens for the frames.

In some embodiments, the at least one processor may be configured to determine a distance between the first landmark associated with the frames for spectacles in the image and a third landmark associated with the frames for spectacles in the image to determine the first dimension.

In some embodiments, the at least one processor may be configured to determine a distance from a camera to the second object and to divide the distance from the camera to the second object by a focal length of the camera to determine the scaling factor.

In some embodiments, the second object may include a pupil.

In some embodiments, the image may be obtained from a camera in communication with the at least one processor. The at least one processor may be configured to segment the first object in the image.

In some embodiments, the image may be a composite image based on a first image obtained from a camera and a model of the first object superimposed on the first image.

### BRIEF DESCRIPTION OF THE FIGURES

The accompanying drawings, which are incorporated herein and form a part of the specification, illustrate the aspects of the present disclosure and, together with the description, further serve to explain the principles of the aspects and to enable a person skilled in the pertinent art to make and use the aspects. The drawings are for illustration purposes only, show exemplary non-limiting embodiments, and are not necessarily drawn to scale.

FIG. 1A is a block diagram of one example of a system in accordance with some embodiments;

FIG. 1B is a block diagram of an example of a computing device that may be used in the system illustrated in FIG. 1A in accordance with some embodiments;

FIG. 2 is one example of a flow diagram of a method in accordance with some embodiments;

FIG. 3A is one example of a flow diagram of a method of computing a distance between objects in an image in accordance with some embodiments;

FIG. 3B is another example of a flow diagram of a method of computing a distance between objects in an image in accordance with some embodiments;

FIGS. 4A-4F are illustrative examples of an image at various intervals of the processes illustrated in FIGS. 3A and 3B in accordance with some embodiments; and

FIGS. 5A-5E are illustrative examples of at least a portion of a graphical user interface (GUI) that may be displayed to a user to allow the user to adjust a location of a landmark and/or to identify a location of a landmark in accordance with some embodiments.

### DETAILED DESCRIPTION

This description of the exemplary embodiments is intended to be read in connection with the accompanying drawings, which are to be considered part of the entire written description. As used herein, use of a singular article such as “a,” “an” and “the” is not intended to exclude pluralities of the article's object unless the context clearly and unambiguously dictates otherwise. The use of the singular includes the plural unless specifically stated otherwise. The use of “or” means “and/or” unless stated otherwise. Furthermore, the use of the term “including,” as well as other forms such as “includes” and “included,” is not limiting. In addition, terms such as “element” or “component” encompass both elements and components comprising one unit, and elements and components that comprise more than one subunit, unless specifically stated otherwise. Additionally, the section headings used herein are for organizational purposes only, and are not to be construed as limiting the subject matter described.

The following description is provided as an enabling teaching of a representative set of examples. Many changes can be made to the embodiments described herein while still obtaining beneficial results. Some of the desired benefits discussed below can be obtained by selecting some of the features discussed herein without utilizing other features. Accordingly, many modifications and adaptations, as well as subsets of the features described herein are possible and can even be desirable in certain circumstances. Thus, the following description is provided as illustrative and is not limiting.

Systems and methods are disclosed herein for determining/obtaining various measurements that may be useful in the field of optometry, among other fields. Examples of such measurements that may be obtained include, but are not limited to, pupillary distance, segment (“seg”) height (e.g., vertical distance between the pupil and the bottom of a lens), custom optical center (“OC”) height (e.g., the vertical distance between the bottom of the lens and a point halfway between the lens's geometric center and the pupil), and monocular PD (e.g., the horizontal distance between each of the pupils and the horizontal midpoint of the glasses). As described herein, the systems and methods may be performed, at least in part, with a computing device, such as a phone, tablet, laptop, desktop, or kiosk, to provide only a few non-limiting examples. The subject may be a mammal or a human, with the mammal or human being male, female, non-binary, or other gender identity, an adult, or a child. The systems and methods advantageously may provide or enable accurate and efficient real-time facial measurements of a subject. The measurements may be stored, such as in a database or other data structure, and associated with an account and/or profile of the user, which may enable the user to receive product recommendations based on facial measurements and/or consumer satisfaction data and/or purchase history associated with historical facial measurements.

### System Overview

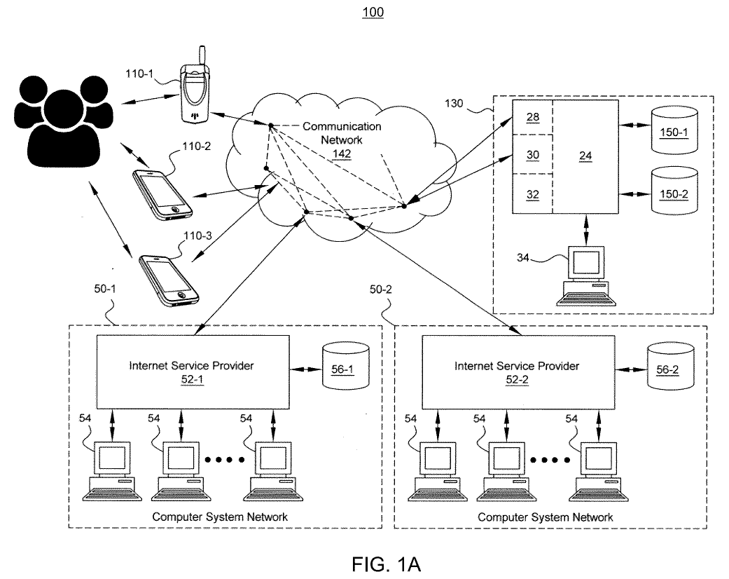

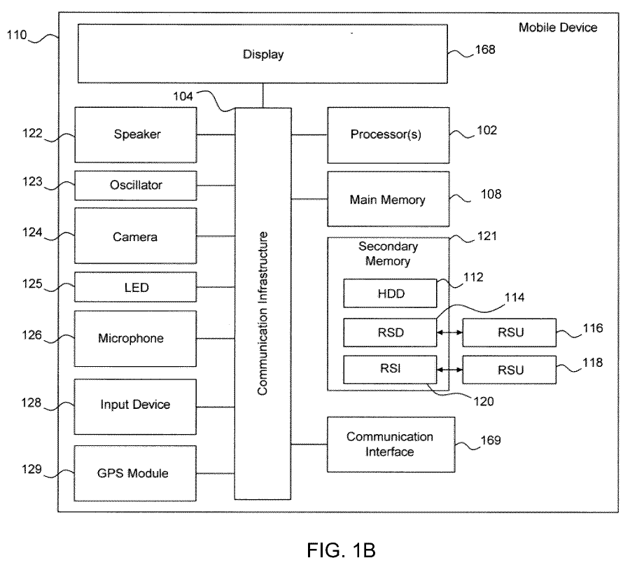

FIG. 1A depicts one example of a system or environment 100 including a plurality of client devices 110-1, 110-2, and 110-3 (collectively, “client devices 110”), which may be communicatively coupled to a to one or more computer system networks 50-1, 50-2 (“computer networks 50”) and to management server 130 via communication network 142. Communication network 142 may be a wide area network (“WAN”), a local area network (“LAN”), personal area network (“PAN”), or other communication network as will be understood by one of ordinary skill in the art. In some embodiments, communication network 142 may be the Internet and client devices 110 may be referred to as being online. For example, “online” may mean connecting to or accessing source data or information from a location remote from other devices or networks coupled to communication network 142.

Management server 130 may include a processing unit 24 coupled to one or more data storage units 150-1, 150-2 (collectively, “data storage units 150” or “data storage 150”). Data may be stored in the data storage units in a structured or unstructured manner. For example, data may be stored in a database, such as a relational and/or sequential database, which may be referred to as a database management system (“DBMS”), or data may be stored in an object store, such as using a hash table and/or an S3 protocol. The processing unit 24 may be configured to provide one or more front-end graphical user interfaces (“GUIs”) (e.g., a user GUI 28 and an advisor GUI 30) and one or more back-end or administrative GUIs or portals 32. The GUIs 28, 30, 32 may be accessible to one or more remote computers 54 and/or to one or more local computers 34. The GUIs or portals 28, 30, 32 can take the form of, for example, a webpage that is displayed using a browser program local to remote computers 54 and/or to one or more local computers 34. It should be understood that the management server 130 may be implemented on one or more computers, servers, or other computing devices. In some embodiments, a GUI may be displayed on client devices 110 via a software application. For example, system 100 may include additional servers programmed or partitioned based on permitted access to data stored in data storage 150. As used herein, “portal” is not limited to general-purpose Internet portals, such as YAHOO!® or GOOGLE®, but also includes GUIs that are of interest to specific, limited audiences and that provide the party access to a plurality of different kinds of related or unrelated information, links and tools as described below. “Webpage” and “website” may be used interchangeably herein.

Remote computers 54 may be part of a computer system network 50-1, 50-2 and may gain access to communication network 142 through an Internet service provider (“ISP”) 52-1, 52-2 (“ISPs 52”). Client devices 110 may gain access to communications network 142 through a wireless cellular communication network, a WAN hotspot, an ISP, and/or through a wired or wireless connection with a computer as will be understood by one skilled in the art. Client users and administrative personnel, as will be described below, may use local computers 34, remote computers 54, and/or client devices 110 to gain access to system 100. Computer system network 50-1, 50-2, may include one or more data storage units 56-1, 56-2 (collectively, “data storage units 56” or “data storage 56”). Data storage units 56 may be similar to the data storage 150 described above, although one of ordinary skill in the art will understand that data storage units 56 may take other forms.

Client devices 110 may be any computing device capable of transmitting and receiving signals via a wired and/or wireless communication medium. Examples of client devices include, but are not limited to, mobile or cellular phones, smart phones, personal digital assistants (“PDAs”), laptop computers, tablet computers, music players, and e-readers, to name a few possible devices.

FIG. 1B is a block diagram of one example of an architecture of a client device 110 in accordance with some embodiments. A client device 110 may include one or more processors, such as processor 102. Processor 102 may be any central processing unit (“CPU”), microprocessor, micro-controller, or computational device or circuit for executing instructions. It should be understood that processor 102 may include multiple processors that perform all or some of the operations in a distributed manner (e.g., in serial and/or parallel). Processor 102 may be connected to a communication infrastructure 104 (e.g., a communications bus, crossover bar (or switch), or network). It should be apparent to one of ordinary skill in the art how to implement the method using client devices 110 that include other systems or architectures beyond that set forth in FIG. 1B. Further, one of ordinary skill in the art will understand that computing devices 34, 54 may have a similar and/or identical architecture as that of client devices 110. Put another way, computing devices 34, 54 may include some, all, or additional functional components as those of the client device 110 illustrated in FIG. 1B.

Client device 110 also may include a display 168 that displays graphics, video, text, and other data received from the communication infrastructure 104 (or from a frame buffer not shown) to a user. Examples of such displays 168 include, but are not limited to, LCD screens, OLED display, capacitive touch screen, and a plasma display, to list only a few possible display types. Client device 110 also may include a main memory 108, such as a random access (“RAM”) memory, and a secondary memory 110. Secondary memory 121 may include a more persistent memory such as, for example, a hard disk drive (“HDD”) 112 and/or removable storage drive (“RSD”) 114, which may represent a magnetic tape drive, an optical disk drive, solid-state drive (“SSD”), or other suitable memory. In some embodiments, removable storage drive 114 may read from and/or write to a removable storage unit (“RSU”) 116 in a manner that is understood by one of ordinary skill in the art. Removable storage unit 116 may represent a magnetic tape, optical disk, or the like, which may be read by and written to by removable storage drive 114. As will be understood by one of ordinary skill in the art, the removable storage unit 116 may include a tangible and non-transient machine-readable storage medium having stored therein computer software and/or data.

In some embodiments, secondary memory 110 may include other devices for allowing executable programs or other instructions to be loaded into client device 110. Such devices may include, for example, a removable storage unit (“RSU”) 118 and a corresponding removable storage interface (“RSI”) 120. Examples of such units 118 and interfaces 120 may include a removable memory chip (such as an erasable programmable read only memory (“EPROM”)), programmable read only memory (“PROM”)), secure digital (“SD”) card and associated socket, and other removable storage units 118 and interfaces 120, which allow software and data to be transferred from the removable storage unit 118 to client device 110.

Client device 110 also may include a speaker 122, an oscillator 123, one or more cameras 124, a light emitting diode (“LED”) 125, a microphone 126, an input device 128, an accelerometer (not shown), and a global positioning system (“GPS”) module 129. Examples of camera 124 features include, but are not limited to optical image stabilization (“OIS”), larger sensors, bright lenses, 4K video, optical zoom plus RAW images and HDR, and “Bokeh mode” with multi lenses and multi-shot night modes, to list a few possible examples. Camera 124 may comprise one or more lenses with different functions. By way of example, camera 124 may include an ultra-wide sensor, telephoto sensor, time of flight sensor, macro sensor, megapixel (“MP”) sensor, and/or a depth sensor. As noted above, camera 124 is not limited to a single camera and includes a camera system that may include multiple different types of cameras, sensors, etc. By way of example, Apple's TrueDepth® camera system may include a front-facing “selfie” camera, an infrared emitter, an infrared camera, a proximity sensor, an ambient light sensor, a flood illuminator, and a dot projector that cooperate to obtain depth map and associated image. In other words, camera 124 of client device 110 may have multiple sensors, cameras, emitters, or other associated components that work as a system to obtain image information for use by client device 110.

Examples of an input device 128 may include, but are not limited to, a keyboard, buttons (physical or virtual), a trackball, voice input (e.g., microphone and corresponding software for recognizing speech), or any other interface or device through which a user may input data. In some embodiments, input device 128 and display 168 may be integrated into the same device. For example, display 168 and input device 128 may be touchscreen through which a user uses a finger, pen, and/or stylus to input data into client device 110.

Client device 110 also may include one or more communication interfaces 169, which allow software and data to be transferred between client device 110 and external devices such as, for example, another client device 110, a computer 34, 54 and other devices that may be locally or remotely connected to client device 110. Examples of the one or more communication interfaces 169 may include, but are not limited to, a modem, a network interface (such as an Ethernet card or wireless card), a communications port, a Personal Computer Memory Card International Association (“PCMCIA”) slot and card, one or more Personal Component Interconnect (“PCI”) Express slot and cards, or any combination thereof. The one or more communication interfaces 169 may also include a wireless interface configured for short range communication, such as near field communication (“NFC”), Bluetooth, or other interface for communication via another wireless communication protocol. As briefly noted above, one of ordinary skill in the art will understand that computing devices 34, 54 and portions of system 100 may include some or all components of client device 110.

Software and data transferred via the one or more communications interfaces 169 may be in the form of signals, which may be electronic, electromagnetic, optical, and/or other signals capable of being received by communications interfaces 169. These signals may be provided to communications interface 169 via a communications path or channel. The channel may be implemented using wire or cable, fiber optics, a telephone line, a cellular link, a radio frequency (“RF”) link, or other communication channels.

In this application, the terms “non-transitory computer program medium” and “non-transitory computer-readable medium” refer to media such as removable storage units 116, 118, and/or a hard disk installed in hard disk drive 112. These computer program products may provide software to client device 110 and/or other computing devices or processors. Computer programs (also referred to as “computer control logic”) may be stored in main memory 108 and/or secondary memory 110. Computer programs may also be received via the one or more communications interfaces 169. Such computer programs, when executed by a processor(s) 102, may enable the client device 110 to perform the features of the methods and systems discussed herein.

In various embodiments, as shown in FIGS. 1A and 1B, client device 110 may include a computing device such as a hashing computer, a personal computer, a laptop computer, a tablet computer, a notebook computer, a hand-held computer, a personal digital assistant, a portable navigation device, a mobile phone, a smart phone, a wearable computing device (e.g., a smart watch, a wearable activity monitor, wearable smart jewelry, and glasses and other optical devices that include optical head-mounted displays (“OHMDs”)), an embedded computing device (e.g., in communication with a smart textile or electronic fabric), or any other suitable computing device configured to store data and software instructions, execute software instructions to perform operations, and/or display information on a display device. Client device 110 may be associated with one or more users (not shown). For example, a user may operate client device 110 causing it to perform one or more operations in accordance with various embodiments.

In embodiments where some or all of the system 100 or methods performed by one or more components of the system 100, which may be partially or entirely implemented using software, the software may be stored in a computer program product and loaded into a computing device 34, 54, a client device 110, and/or management server 130 using removable storage drive, hard drive, and/or communications interface. The software, when executed by one or more processor, causes the one or more processor to perform the functions of the method described herein. In some embodiments, the methods may be implemented primarily in hardware using, for example, hardware components such as application specific integrated circuits (“ASICs”). Implementation of the hardware state machine to perform the functions described herein will be understood by persons skilled in the art. In some embodiments, the methods may be implemented using a combination of both hardware and software.

Embodiments of the subject matter described in this specification can be implemented in a system 100 that includes a back end component, e.g., as a data server, or that includes a middleware component, e.g., an application server, or that includes a front end component (e.g., a client device 110) having a graphical user interface or a Web browser through which a user can interact with an implementation of the subject matter described is this specification, or any combination of one or more such back end, middleware, or front end components. The components of the system can be interconnected by any form or medium of digital data communication, (e.g., a communication network 142). Communications network 142 may include one or more communication networks or media of digital data communication. Examples of communication network 142 include a local area network (“LAN”), a wireless LAN (e.g., a “WiFi” network), a RF network, a NFC network, a wireless Metropolitan Area Network (“MAN”) connecting multiple wireless LANs, NFC communication link(s), and a wide area network (“WAN”), e.g., the Internet and combinations thereof. In accordance with various embodiments, communications network 142 may include the Internet and any publicly accessible network or networks interconnected via one or more communication protocols, including, but not limited to, HyperText Transfer Protocol (“HTTP”), HyperText Transfer Protocol Secured (“HTTPS”), Secured Socket Layer/Transport Layer Security (“SSL/TLS”), and transmission control protocol/internet protocol (“TCP/IP”). Communications protocols in accordance with various embodiments also include protocols facilitating data transfer using radio frequency identification (“RFID”) communications and/or NFC. Moreover, communications network 142 may also include one or more mobile device networks, such as a GSM or LTE network or a PCS network, allowing a client device to send and receive data via applicable communications protocols, including those described herein. For ease of illustration, communication network 142 is shown as an extension of management server 130.

A client device 110 and management server 130 are generally remote from each other and typically interact through a communication network 142. The relationship of client device 110 and management server 130 may arise by virtue of computer programs running on the respective system components and having a client-server relationship to each other. System 100 may include a web/application server (not shown) in embodiments used to gain access to many services provided by management server 130.

In some aspects, a client device 110 may store in memory one or more software applications that run on the client device when executed by the one or more processors 102 of the client device 110. In some instances, each client device 110 may store software applications that, when executed by one or more processors 102, perform operations that establish communications with management server 130 (e.g., across communication network 142 via communication interface 169) and that obtain, from management server 130, information or data from the data stores 150 in accordance with various embodiments.

In various embodiments, client device 110 may execute stored software application(s) to interact with management server 130 via a network connection. The executed software applications may cause client device 110 to communicate information (e.g., facial measurements, user profile information, device information, and/or other information). As described below, executed software applications(s) may be configured to allow a user associated with client device 110 to obtain one or more measurements using a camera 124. Stored software application(s) on client device 110 may be configured to access webpages on the Internet or other suitable network based communication capable of interacting with communication network 142, as will be understood by one of ordinary skill in the art. For example, a user may access a user account on management server 130 via an Internet webpage. The management server 130 may be configured to render the Internet webpage for the user on client device 110. Alternatively or additionally, management server 130 may provide information to stored software application(s) on client device 110 via communication network 142. The client device 110 may display information provided by management server 130 using a stored software application(s) graphical user interface display. A respective user account may be associated with a developer, client user, or supervisor/monitoring authority as will be understood by one of ordinary skill in the art and described below.

As noted above, system 100 may include data stores 150, which may be configured to store and manage data, such as facial measurement information (e.g., PD, segment (“seg”) height, OC height, etc.), user account authentication information, and other data maintained by the management server 130. The data stores 150 may be communicatively coupled with various modules and engines as will be understood by one of ordinary skill in the art.

It should be understood that various forms of data storage or repositories can be used in system 100 that may be accessed by a computing system, such as hard drives, tape drives, flash memory, random-access memory, read-only memory, EEPROM storage, in-memory databases like SAP HANA, and so on, as well as any combination thereof. Stored data may be formatted within data stores in one or more formats, such as flat text file storage, relational databases, non-relational databases, XML, JSON, comma-separated values, Microsoft Excel files, or any other format known to those of ordinary skill in the art, as well as any combination thereof as is appropriate for the particular use. Data stores may provide various forms of access to the stored data, such as by file system access, network access, S3, a SQL protocol (e.g., ODBC), HTTP, FTP, NES, CIFS, and so on, as well as any combination thereof.

According to various embodiments, client device 110 may be configured to access data storage 150 via management server 130. In various embodiments, the data storage 150 may be configured to maintain a database schema. For example, a database schema may be arranged to maintain identifiers in columns within data storage 150 associated with facial measurement or user account information. In this aspect, identifiers may refer to specific information pertaining to the categories described above. A database schema within the data storage 150 may be arranged or organized in any suitable manner within the system. Although the above-described examples identify categorical identifiers, any number of suitable identifiers may be used to maintain records associated with the system described herein. In addition, a database schema may contain additional categories and identifiers not described above for maintaining record data in system 100. The database can also provide statistics and marketing information associated with users of system 100. A database schema may advantageously organize identifiers in a way that permits the system to operate more efficiently. In some embodiments, categories of identifiers in the database schema increase efficiency by grouping identifiers with an associated management model of management server 130.

In some embodiments, data may be stored as a block or blob in an object store that is accessible using one or more identifiers. For example, the object may include various types of information associated with a user and may be accessed using a hash or other unique (or substantially unique) value. One of ordinary skill in the art will understand that the data storage 150 may be configured in a number of ways to facilitate data storage and retrieval.

In various embodiments, management server 130 may include computing components configured to store, maintain, and generate data and software instructions. For example, management server 130 may include or have access to one or more processors 24, one or more servers (not shown) and tangible, non-transitory memory devices for storing software or code for execution and/or additional data stores. Servers may include one or more computing devices configured to execute software instructions stored on to perform one or more processes in accordance with various embodiments. In some embodiments, data storage 150 may include or be associated with a server that executes software instructions to perform operations that provide information to at least one other component of computing environment 100, for example providing data to another data store or to third party recipients (e.g., banking systems, third party vendors, information gathering institutions, etc.) through a network, such as a communication network 142.

Management server 130 may be configured to provide one or more websites, digital portals, or any other suitable service that may be configured to perform various functions of management server 130 components. In some embodiments, management server 130 may maintain one or more application programming interfaces (“APIs”) through which the functionality and services provided by server 130 may be accessed through one or more application programs executed by a computing device 34, 54, 110. In various embodiments, management server 130 may provide information to software application(s) on client device 110 for display on a graphical user interface 168.

In some embodiments, management server 130 provides information to client device 110 (e.g., through the API associated with the executed application program). Client device 110 presents portions of the information to corresponding users through a corresponding respective graphical user interface 168 or webpage.

In various embodiments, management server 130 may be configured to provide and/or receive information associated with services provided by management server 130. For example, client device 110 may receive information from management server 130 via communication network 142, which may be stored in a locally accessible store device and/or network-accessible storage devices and data stores (e.g., cloud-based storage). For example, client device 110 may execute stored instructions (e.g., an application program, a web browser, and/or a mobile application) to process portions of stored data and render portions of the stored data for presentation to the respective user or users. Management server 130 may include additional servers (not shown) which may be incorporated as a corresponding node in a distributed network or as a corresponding networked server in a cloud-computing environment. Furthermore, servers may communicate via communication network 142 with one or more additional servers (not shown), that may facilitate the distribution of processes for parallel execution by the additional servers.

### Application Overview

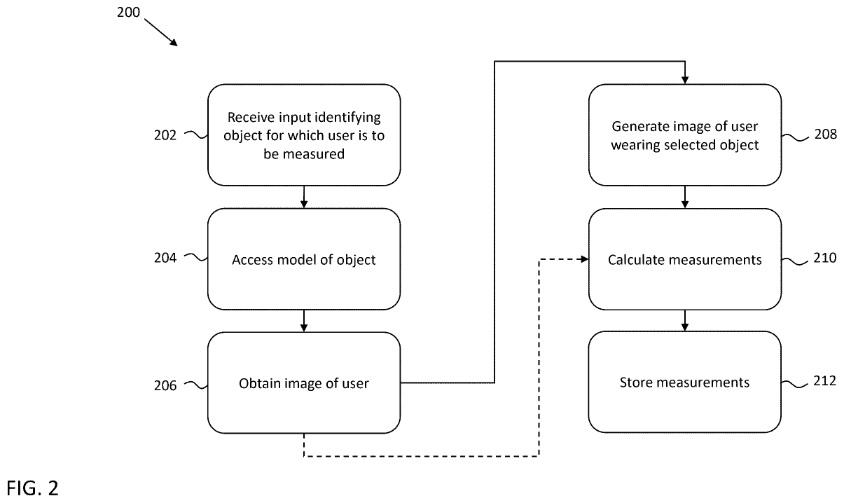

FIG. 2 is a flow diagram of one example of an application that may be run by a computing device such as a computer 34, 54 and/or a client device 110. Although the application is described as obtaining various measurements for fitting a person's face for glasses or spectacles, it should be understood that the application may be used to obtain measurements for a variety of reasons beyond those described herein.

At block 202, a selection may be received. In some embodiments, the selection identifies an object, such as a pair of spectacles, for which the user would like to be measured. Although spectacles (i.e., frame and lenses) are used in the following description, it should be understood that the following description may be used to measure a frame for spectacles or other object. The selection may be made by a user using an input device 128. For example, a user may access a webpage via a webpage or start an application (e.g., an “app”) on the computing device, such as a computer 34, 54 or client device 110. The webpage or app may access a local or remote data store of various spectacles that may be available. The spectacles may be offered for purchase by a retailer or a third-party as will be understood by one of ordinary skill in the art. The user may select one or more of the available spectacles (or other objects) presented to the user on a display 168. In some embodiments, the user may already be in possession of the spectacles and may access the app to perform one or more measurements discussed herein.

At block 204, a model of the selected object may be accessed. For example, if the model of the identified or selected object is stored locally, then the model may be retrieved from memory. If the model of the identified or selected object is not available locally, then a model of the object may be retrieved from a remote data storage, such as data storage 150. As described above, data storage 150 may be accessible via communication network 142 and may be accessible by making a call to management server 130. In some embodiments, the model of the object, e.g., spectacles, may be accessed even if the user is in possession of the spectacles. However, in some embodiments, the process performed at block 204 may be omitted if the user is in possession of the object. For example, the user may be queried as to whether the user is in possession of the object and the app may skip accessing a model of the object if the user responds to the query in the affirmative, such as by inputting a response using an input device 128.

At block 206, an image may be obtained. The image may be obtained using a camera 124 and include all or part of a user's face or head. The camera 124, which may include one or more cameras or image sensors, may be capable of obtaining a two-dimensional (“2D”) or three-dimensional (“3D”) image of the user. As discussed above, the camera 124 may be part of or communicatively coupled with a computing device, such as a computer 34, 54 or client device 110. In some embodiments, the app may guide or prompt the user to position the camera 124 in a desired position, such as described in U.S. Provisional Patent Application No. 63/359,311, filed Jul. 8, 2022, and U.S. patent application Ser. No. 18/346,948, filed Jul. 5, 2023, both entitled “Image Capture Devices, Systems, and Methods,” the entireties of which are incorporated by reference herein. However, in some embodiments, the image may be obtained by using a rear-facing camera and the user taking a “selfie” with little or no prompting by the app. One of ordinary skill in the art will understand that one or more images may be obtained using other methods or techniques. If the user is in possession of the object, then the user may be wearing the object when the image is obtained.

At block 208, a composite image may be generated of the object placed on the user in the obtained image. For example, the model of the object accessed at block 204 may be used such that the object is superimposed on the user in the image of the user obtained at block 206 such that the generated image is a composite image. The placement of the object on the user may be performed by the app using one or more of the methods disclosed in U.S. Pat. No. 10,825,260 and/or U.S. patent application Ser. No. 17/856,482, filed Jul. 1, 2022, for example. One of ordinary skill in the art will appreciate that the object may be placed (or superimposed) on the user using other methods.

In some embodiments, the user or other individual may be able to adjust a position of the object on the user in a GUI displayed to the user or other individual. For example, the user may be able to use an input device 128 to move the object (e.g., spectacles) relative to the user (e.g., a face of the user) in the image. Such movement may include horizontal and/or vertical movement as will be understood by one of ordinary skill in the art.

In some embodiments, an individual other than the user may be provided with the ability to adjust the object in relation to the user. For example, an advisor, which may be a sales associate, optometrist, optician, or other individual located remotely from the user, may be able to adjust the object relative to the user by using a separate computing device, such as a remote computer 34, 54 or a mobile device 110. In some embodiments, the obtained image and the associated model may be made available to the remotely located advisor via the communication network 142. For example, the obtained image may be uploaded to the management server 130, which may be accessible to the advisor via a portal or GUI (e.g., advisor GUI or portal 30). The advisor may be able to adjust the object relative to the user using an input device 128 in a manner similar to that described above with respect to the user making the adjustments.

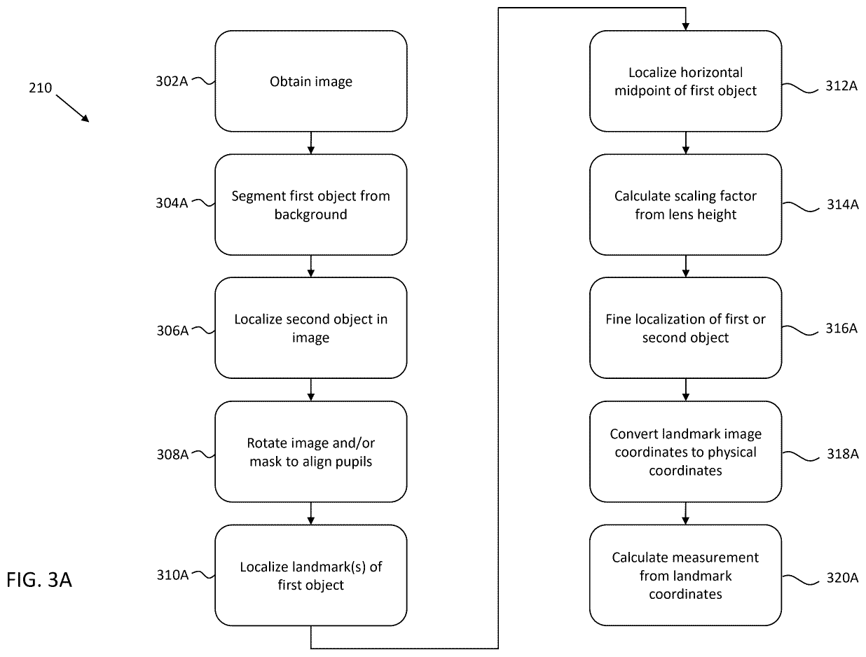

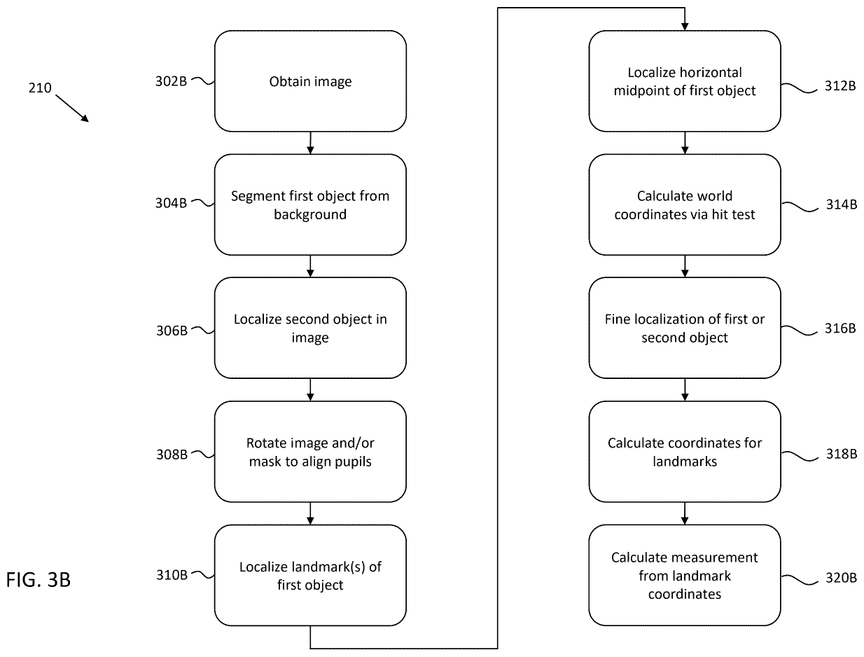

At block 210, one or more measurements may be calculated. The measurements may be calculated using one or more of the processes described below with reference to FIGS. 3A-5E.

At block 212, the one or more measurements may be stored. In some embodiments, the one or more measurements may be stored locally, such as in main memory 108 and/or in secondary memory 121 of client device 110. Additionally or alternatively, the one or more measurements may be stored remotely from client device, such as in a data store 150 associated with management server 130. The one or more measurements may be stored such that the measurements are associated with a user or a profile of a user, or a spectacle order.

### Measurement Calculation(S)

FIG. 3A is a flow diagram of one example of a process for calculating one or more measurements between objects in an image in accordance with some embodiments. The process shown in FIG. 3A may be performed at block 210 during the execution of the method 200 shown in FIG. 2. As described above, the process may be performed locally on a computing device, such as a computer 34, 54 or mobile device 110. In some embodiments, the process may be performed at the management server 130. In some embodiments, the process may be performed in a distributed fashion using multiple computing devices, which may be located remotely from one another and communicatively coupled via communication network 142. Although the method of calculating a measurement is described as being used to determine one or more distances between a physical landmark, such as a pupil, and an area of a wearable object, such as an edge or center of a lens or frame, it should be understood that the method may be used to calculate one or more distances between other objects in an image.

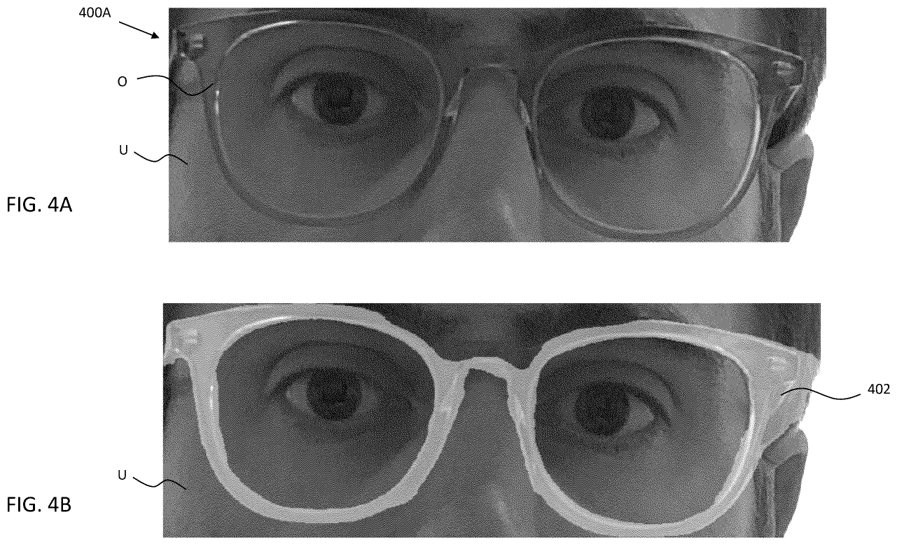

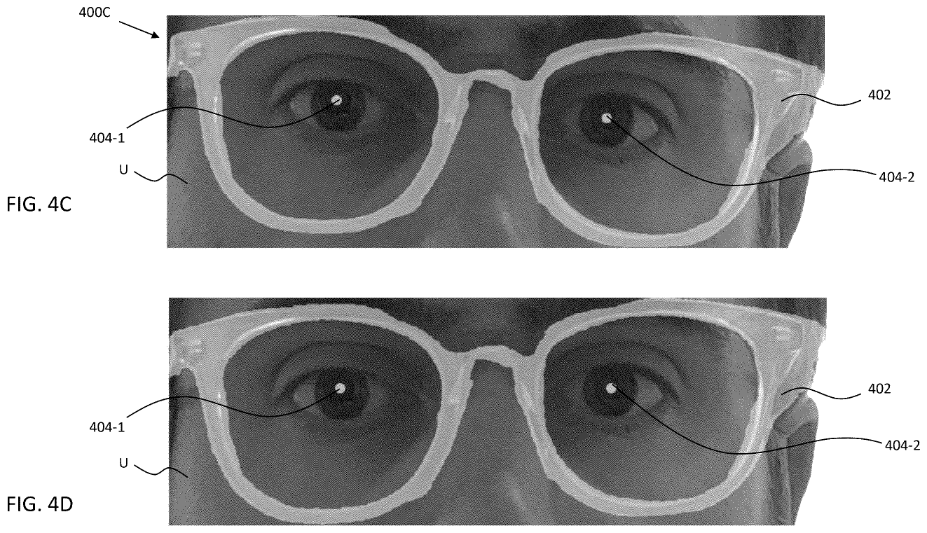

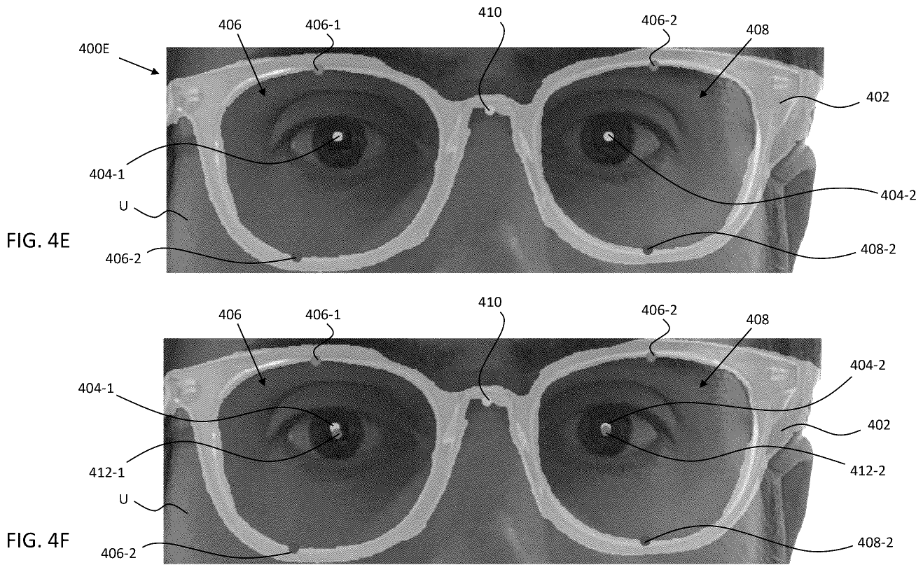

At block 302A, the obtained image is accessed. In some embodiments, the obtained image is the image generated at block 208 of FIG. 2, which includes the model of the object disposed on the user in the image. In some embodiments, the obtained image is the image obtained at block 206 in FIG. 2 if the user is wearing the object when the image is taken, as described above. The image may be a 2D image having 2D image data (e.g., one or more X and Y values, which may correspond to a pixel), although other image types (e.g., a 3D image) may be used. FIG. 4A illustrates one example of an image 400A in which a user U is wearing an object O.

At block 304A, the image may be segmented to identify the object in the image. For example, in embodiments in which the object O is a pair of spectacles being worn by a user U in the image, such as shown in FIG. 4A, the frame of the spectacles may be detected and then segmented from the background. In some embodiments, the object may be detected using a YoloV4-tiny neural network, although one ordinary skill in the art will understand that the object may be detected in the image using other neural networks and/or modules. The segmentation may be performed using a UNet convolutional neural network with a ReLU activation function, as described in Ronneberger et al., “U-Net: Convolutional Networks for Biomedical Image Segmentation,” which is incorporated by reference herein in its entirety. Again, it should be understood that other neural networks, modules, or processes may be used to perform the segmentation. FIG. 4B illustrates an example of an image 400B after the detection and segmentation of the object O has been performed. As shown in the representation illustrated in FIG. 4B, the object (e.g., frame) has been detected and a mask 402 applied to the object.

In some embodiments, the segmentation may be omitted. For example, if a model of an object O is placed on the user U in the image (i.e., the user U was not wearing the object O when the image was acquired and thus the image is a composite image), then the segmentation process may not be performed because the model of the object and the underlying image are separate from one another. Consequently, the boundaries of the object O may be known without performing segmentation, and thus the segmentation process may be omitted.

At block 306A, a second object may be identified or localized in the image. For example, in some embodiments, one or more pupils may be localized in the image, although other landmarks or objects may be identified or localized as will be understood by one of ordinary skill in the art. In some embodiments, the second object may be localized using Apple's Vision Framework, although other processing modules or algorithms may be used to identify the second object in the image. In some embodiments, Apple's CoreML may be used to apply one or more models to the image. FIG. 4C illustrates a representation of the image 400C in which the first object O (e.g., frame) has been segmented and a second object (e.g., pupils) 404-1, 404-2 have been localized. Further examples of identifying and/or localizing pupils in an image are described in U.S. Patent Application Publication No. 2021/0393121.

At block 308A, the image may be rotated. The rotation of the image may be performed to align the alignment of the second object in a certain direction. For example, the image may be rotated to align the pupils 404-1, 404-2 horizontally as shown in FIG. 4D. It should be understood that the image may be rotated in either of a clockwise direction or a counter-clockwise direction to align the second objects. It should further be understood that while the objects may be aligned in a horizontal axis, the second object(s) may be aligned in other directions (e.g., vertically, diagonally, etc.).

At block 310A, one or more landmarks of the first object may be identified and/or localized in the image. The number and type of the landmarks that may be identified and/or localized may vary. In the example illustrated in FIG. 4E, the points 406-1, 406-2 of lens 406 and the points 408-1, 408-2 of lens 408 may be identified and/or localized. The landmarks of the second object, e.g., points 406-1, 406-2, 408-1, 408-2 of the lenses 406, 408, may be determined, at least in part, from the segmentation mask 402. The identification and/or localization of the landmarks may be determined using OpenCV's connectedComponents ( ) function, although other functions and modules may be used. In some embodiments, the top points 406-1, 408-1 and bottom points 406-2, 408-2 of lenses 406, 408 may be determined by identifying the uppermost and lowermost points in components encompassing the pupils 404.

At block 312A, a midpoint of the first object may be identified and/or localized. Still referring to the example illustrated in FIG. 4E, a horizontal center 410 of the frame, as identified by the mask 402, may be identified. In some embodiments, the horizontal center 410 may be determined by finding a vertical line about which the mask 402 is symmetric (e.g., axis or line of symmetry). For example, symmetry may be assessed by applying a binary AND operation to a portion of the mask 402 to the left of a candidate line of symmetry and to the right of the line of symmetry. A global sum of the result may be calculated. A dividing line that yields the largest sum may be identified as the horizontal center of the mask 402.

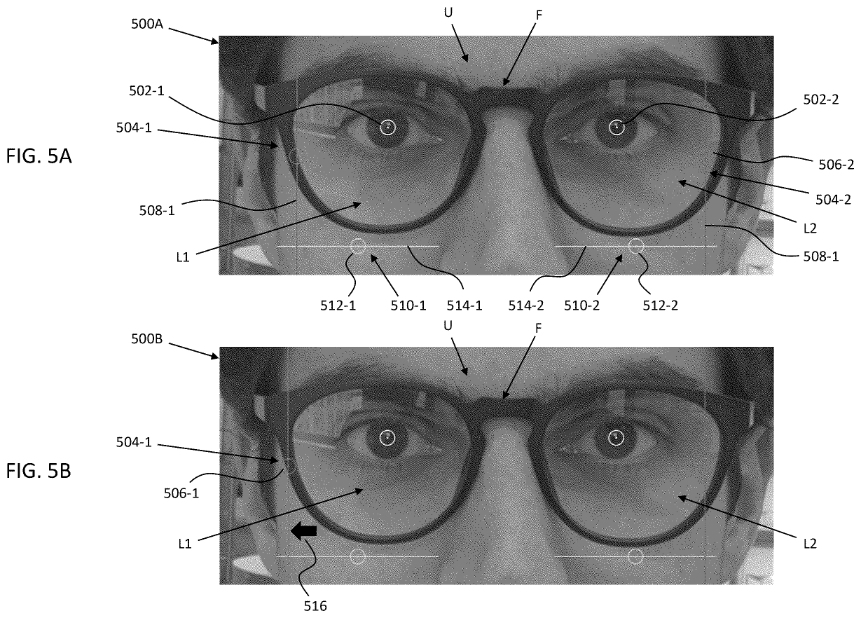

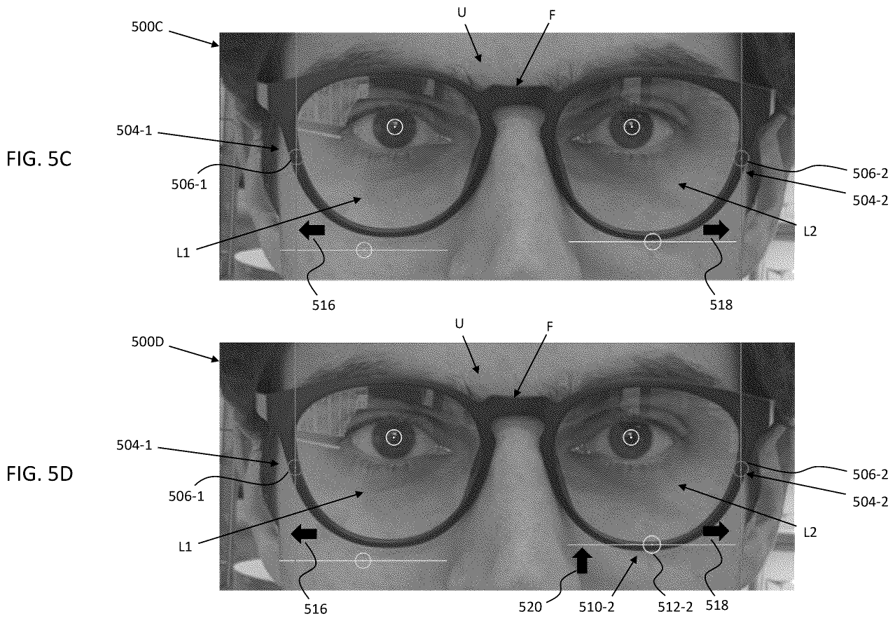

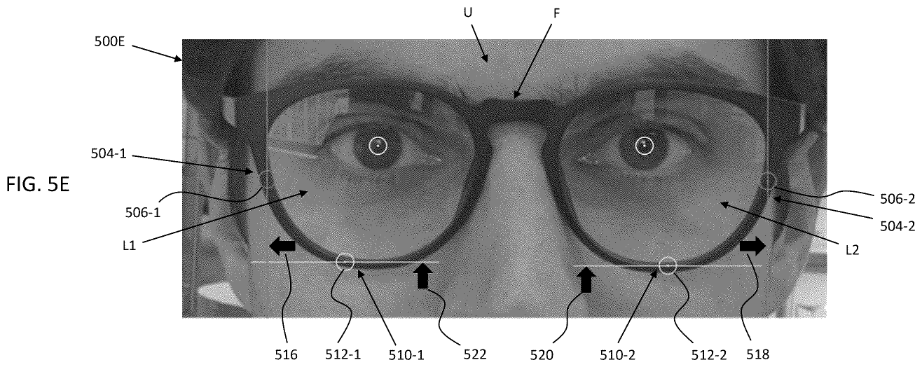

While the processes of blocks 310A and 312B may be performed in a purely automated process, i.e., entirely by one or more processors, the localization of landmarks and/or midpoint of objects may be performed with manual assistance. For example, in some embodiments, a user or other individual may be provided with the ability to adjust the location of the landmark(s) and/or the midpoints of the object in the image. User input may be received via a GUI in a similar manner as that described above with respect to block 208 and the placement and/or adjustment of the object on the face of the user in the image. FIGS. 5A-5E illustrate an example of at least a portion of a GUI 500 (shown at different points of manipulation identified as 500A, 500B, 500C, 500D, 500E) with which a user or other individual may interact to adjust and/or identify the locations of the landmarks and/or midpoints of an object. In the example shown in FIGS. 5A-5D, the landmarks are part of a frame F of spectacles having lenses L1 and L2 being worn by a user U. However, as noted above, the landmarks could be part of other objects, including part of the lenses L1 and L2, for example.

Referring first to FIG. 5A, the GUI 500A displays an image of the user U wearing an object, i.e., frames F for eyeglasses having lenses L1, L2. The pupils of the eyes of the user U may be identified in the image by highlighting (e.g., a solid circle) and/or with concentric circles 502-1, 502-2, as shown, although indicia or visual cues may be used. The pupils may be previously identified or localized during step 306A in FIG. 3A, as discussed above. The GUI 500A may also include one or more first sliders 504-1, 504-2 (collectively, “first sliders 504”) and one or more second sliders 510-1, 510-2 (collectively, “second sliders 510”).

In some embodiments, the first sliders 502 may be configured to move in a first direction, which may be a horizontal direction, and the second sliders 510 may be configured to move in a second direction, which may be a vertical direction or a direction different from the first direction. Each of the first sliders 504-1, 504-2 may include a respective locating circle 506-1, 506-2 (collectively, “locating circles”) and one or more respective vertically extending guide bar portions 508-1, 508-2 (collectively, “vertical guide bars 508”). Each of the second sliders 510-1, 510-2 may include a respective locating circle 512-1, 512-2 (collectively, “locating circles 512”) and one or more respective horizontally extending guide bar portions 514-1, 514-2 (collectively, “horizontal guide bars 514”). In some embodiments, the locating circles 506 may be able to be moved along the vertical guide bars 508, and locating circles 512 may be able to be moved along the horizontal guide bars 514.

A user or other individual may position and/or reposition the one or more first sliders 504 and/or the one or more second sliders 510 such that the locating circles 506, 512 are located over a landmark of the object, e.g., frame F. As will be understood by one of ordinary skill in the art, the user or other individual may use a finger, stylus, physical key on a keyboard, mouse, or other user input means to reposition the one or more first sliders 504 and one or more second sliders 510. In some embodiments, the user or other individual may be prompted to reposition the one or more first sliders 504 and the one or more second sliders 510 in a predetermined order, although the user or other individual may be allowed to reposition the one or more first sliders 504 and the one or more second sliders 510 in any order the user or other individual desires. The user inputs (e.g., manipulation of the first and/or second sliders 504, 510) may be converted to signals that may be interpreted by the processor(s) 102 as will be understood by one of ordinary skill in the art.

For example, FIG. 5B shows a GUI 500B after a user or other individual has moved first slider 504-1 in a direction identified by arrow 516 to locate the locating circle 506-1 over a landmark, such as a middle of an outer portion of the frame F surrounding lens L1. FIG. 5C shows a GUI 500C after a user or other individual has moved first slider 504-1 in a direction identified by arrow 516 and first slider 504-2 in a direction identified by arrow 518. In some embodiments, first slider 504-2 is moved such that locating circle 506-2 identifies the location of a landmark, which in this example may be a middle of an outer portion of the frame F surrounding lens L2. As noted above, the order in which the sliders 504-1, 504-2 are repositioned by a user or other individual may be predetermined or selected by the user or other individual.

FIG. 5D illustrates one example of a GUI 500D after a user or other individual has moved first slider 504-1 in a direction identified by arrow 516, first slider 504-2 in a direction identified by arrow 518, and second slider 510-2 in a direction identified by arrow 520. In some embodiments, the second slider 510-2 is moved such that locating circle 512-2 identifies the location of a landmark, which in this example may be a middle of a bottom portion of the frame F surrounding lens L2. FIG. 5E illustrates one example of a GUI 500E after a user or other individual has moved first slider 504-1 in a direction identified by arrow 516, first slider 504-2 in a direction identified by arrow 518, second slider 510-2 in a direction identified by arrow 520, second slider 510-1 in a direction identified by arrow 522, and second slider 510-1 in a direction identified by arrow 524. In some embodiments, second slider 510-1 is moved such that locating circle 512-1 identifies the location of a landmark, which in this example may be a middle of a bottom portion of the frame F surrounding lens L1. As noted above, the order in which the sliders 504-1, 504-2, 510-1, 510-2 are repositioned by a user or other individual may be predetermined or selected by the user or other individual.

Referring again to FIG. 3A, at block 314A, a scaling factor is determined or calculated. In some embodiments, a scaling factor may be determined in order to provide a relative scale between a first unit of measure (e.g., millimeters, centimeters, inches, or any other physical unit of measurement) and a second unit of measure (e.g., pixels or other virtual unit of measurement). The scaling factor may be determined by receiving a lens height or “B” value, which may be provided in a first unit of measure (e.g., millimeters, centimeters, inches, etc.). The B value may be input or received in a variety of ways. For example, in some embodiments, the lens height (e.g., vertical distance) may be manually entered by a user or an advisor using an input device 128. Additionally or alternatively, the lens height (or other known dimension) may be accessed from a model, such as a 2D or 3D model, or from a data storage, such as data storage 150.

To determine the scaling factor, a measurement of an object that corresponds in the image to the B value may be determined. For example, if the B value corresponds to the lens height, then the lens height in the image may be determined in a second unit of measure (e.g., pixels). The calculated distance, which is designated as CD and in a unit of pixels, may be set as the numerator in the following equation for calculating the scaling factor in a two-dimensional image, SF_{2D}:

$$S {F}_{2 D} = \frac{C D}{B} . \qquad Eq . 1$$

The scaling factor SF_{2D} computed in accordance with Equation 1 may thus be used to convert a measurement or value in a first unit of measure (e.g., millimeters, centimeter, inches, etc.) to a second unit of measure (e.g., pixels).

At block 316A, the process may perform fine object localization. In some embodiments, the fine object localization is performed using one or more of the methods described in U.S. Patent Application Publication No. 2021/0393121. An iris may be assumed to have a diameter of approximately 12 mm, which may be used as an initial value to determine the approximate distance of the eye from the camera 124. It should be understood that the method described herein may be used to obtain other measurements using other objects as a reference, and thus other initial approximations may be used for an object.

The diameter of the iris in pixels, A pixel diameter D_{P} of an iris (or other object) may be predicted using the following equation:

$${D}_{P} = ( \frac{f}{z} ) d$$

where,
f is the focal length of the camera;
z is the distance between the camera and the pupil; and
d is the iris diameter.

### Eq. 2

The fine localization may include centering a 1.5D_{P} circle around the object (e.g., an iris) using the initial position of the object as determined by the localization process performed at block 306A. In some embodiments, horizontal gradient kernels may be applied to identify areas with the strongest dark-to-light transitions, which may correspond to the periphery or boundary of the object (e.g., iris). In some embodiments, only select areas of the circle may be considered to account for instances when the object may be occluded. For example, because an entire iris may not be visible, areas that are less than 30° up from the center of the circle and less than 45° down from the center of the circle may be considered. It should be understood that other fine localization processes may be performed. FIG. 4F illustrates one example of an image showing an initial identification of the pupils 404-1, 404-2, such as after the initial localization performed at block 306A, and the pupils 412-1, 412-2 after the fine object localization performed at block 316A.

At block 318A, landmark image coordinates may be converted to physical coordinates. For example, the scale factor SF_{2D} may be used to convert the coordinates measured in pixels to another unit of measure (e.g., millimeters, centimeters, inches, etc.), as will be understood by one of ordinary skill in the art. Thus, the coordinates of the landmarks, such as the landmarks shown in FIGS. 4A-4F and FIGS. 5A-5E, may be converted from pixels to another unit of measure.

At block 320A, one or more measurements may be calculated from the landmark coordinates. Various measurements may be calculated using the determined landmarks. For example, left and right seg heights and left and right PDs. Further, there may be multiple ways of computing the various measurements. For example, one of the following equations may be used to determine various seg heights (“SH”):

$${SH}_{1} = pupil height - lens bottom . \qquad Eq . 3$$

$${SH}_{2} = B - ( lens top - pupil height ) . \qquad Eq . 4$$

$${SH}_{3} = \frac{( {SH}_{1} + {SH}_{2} )}{2} . \qquad Eq . 5$$

$${SH}_{4} = B \times \frac{( pupil height - lens bottom )}{( lens top - lens bottom )} . \qquad Eq . 6$$

In some embodiments, the custom OC height (“COC”) may be determined as the vertical distance between the bottom of a lens (e.g., lens 406 or lens 408) and the halfway point between the lens's geometric center and the pupil using the following equation:

$$C O C = \frac{S H}{2} + \frac{B}{4} . \qquad Eq . 7$$

The PD values may be determined from the horizontal distance between the pupils and the horizontal frame. In some embodiments, measurements obtained from individual image frames may be aggregated together to obtain measurements with lower variability. It should be understood that such aggregation may be performed for measurement values other than PD values, such as seg height and/or COC height, for example. The measurement values may be stored locally or in a remote data storage as block 212 in FIG. 2. For example, in some embodiments, the measurement values may be saved in a data store 150 at or in communication with management server 130 such that the measurement values are associated with a profile of the user.

FIG. 3B illustrates another example of a process for calculating one or more measurements between objects in an image in accordance with some embodiments. Like the process described above with reference to FIG. 3A, the process illustrated in FIG. 3B may be performed at block 210 during the execution of the method 200 shown in FIG. 2. The process shown in FIG. 3B, like the process described above with reference to FIG. 3A, may be performed locally on a computing device, such as a computer 34, 54 or mobile device 110. In some embodiments, the process may be performed at the management server 130. In some embodiments, the process may be performed in a distributed fashion using multiple computing devices, which may be located remotely from one another and communicatively coupled via communication network 142. Although the method of calculating a measurement is described as being used to determine one or more distances between a physical landmark, such as a pupil, and an area of a wearable object, such as an edge or center of a lens, it should be understood that the method may be used to calculate one or more distances between other objects in an image.

At block 302B, the obtained image is accessed. In some embodiments, the obtained image is the composite image generated at block 208 of FIG. 2, which includes the model of the object O disposed on the user U in the image. In some embodiments, the obtained image is the image obtained at block 206 in FIG. 2 if the user is wearing the object when the image is taken, as described above. The image may be a 3D image obtained using a 3D camera or scanner (e.g., one or more cameras imaging devices that are configured to generate an image in which each pixel includes three-dimensional image data values, such as X, Y, and Z), although other image types (e.g., a 3D image) may be used. As will be understood by one of ordinary skill in the art, a 3D image may include 2D image data and a corresponding depth value for each of the pixels in the 2D image data. Such images may be obtained using a variety of devices, including a range camera, a stereo camera, a 3D scanner, and a time-of-flight camera, to list only a few possibilities. FIG. 4A illustrates one example of an image 400A in which a user U is wearing an object O.

At block 304B, the image may be segmented to identify the object in the image. The segmentation process performed at block 304B may be the same process described above with respect to FIG. 304A. For example, an object O, such as a frame for spectacles worn by a user U, may be detected using a YoloV4-tiny neural network, although one ordinary skill in the art will understand that the object O may be detected in the image using other neural networks and/or modules. The segmentation may be performed using a UNet convolutional neural network with a ReLU activation function, although neural networks, modules, or processes may be used to perform the segmentation. The segmentation may yield an image 400B in which the frames of the spectacles are identified or covered by a mask 402.

In some embodiments, as described above with respect to block 304A, the segmentation may be omitted. For example, if a model of an object O is placed on the user in a composite image (i.e., the user U was not wearing the object O when the image was acquired), then the segmentation may not be performed because the model of the object and the rest of image are separate from one another. Consequently, the boundaries of the object O may be known and the segmentation process omitted.

At block 306B, a second object may be identified or localized in the image. Here again, the functions performed at block 306B may be the same as those described above with respect to block 306A. For example, in some embodiments, one or more pupils may be localized in the image using Apple's Vision Framework, although other processing modules or algorithms may be used to identify a second object in the image. As described above, FIG. 4C illustrates a representation of the image 400C in which the first object (e.g., frame) has been segmented and a second object (e.g., pupils) 404-1, 404-2 have been localized. Similarly, FIG. 5A illustrates an example of a GUI in which the pupils of the user U have been identified as indicated by circles 502-1, 502-2.

At block 308B, the image may be rotated. As described above with reference to block 308A, the rotation of the image may be performed to align the alignment of the second object in a certain direction. For example, the image may be rotated, such as in a counterclockwise or clockwise direction, to align the pupils 404-1, 404-2 horizontally as shown in FIG. 4D, although the image may be rotated in other directions. It should be understood that the image may be rotated to align objects in other orientations (e.g., vertically, diagonally, etc.).

At block 310B, one or more landmarks of the first object may be identified and/or localized in the image. As described above, the identification and/or localization of the landmarks may be determined using OpenCV's connectedComponents( ) function, although other functions and modules may be used. Further, the number and type of the landmarks that may be identified and/or localized may vary. In the example illustrated in FIG. 4E, the points 406-1, 406-2 of lens 406 and the points 408-1, 408-2 of lens 408 may be identified and/or localized. The landmarks of the second object, e.g., points 406-1, 406-2, 408-1, 408-2 of the lenses 406, 408, may be determined, at least in part, from the segmentation mask 402. In some embodiments, the top points 406-1, 408-1 and bottom points 406-2, 408-2 of lenses 406, 408 may be determined by identifying the uppermost and lowermost points in components encompassing the pupils 404.

It should be understood that other landmarks, such as edges and/or midpoints of the frame F (e.g., an object O) may be used as illustrated in FIGS. 5A-5E as described above. As described above with respect to FIGS. 5A-5E, a user or other individual may be enabled to adjust the landmark location of one or more of the landmarks identified via the automatic process described above or the user or other individual may be enabled to identify the landmark location using the one or more first sliders 502 and/or one or more second sliders 510.

At block 312B, a midpoint of the first object may be identified and/or localized. For example, a horizontal of center 410 of the frame, which is covered by a mask 402 in the image 400E shown in FIG. 4E, may be identified. As described above with respect to FIG. 312A, the horizontal center 410 may be determined by finding a line of symmetry, which may be done by applying a binary AND operation to a portion of the mask 402 to the left of a candidate line of symmetry and to the right of the line of symmetry. A global sum of the result may be calculated, and a dividing line that yields the largest sum may be identified as the horizontal center of the mask 402.

At block 314B, world coordinates may be calculated. First, known intrinsic parameters of the camera, such as the focal length and principal point, may be used to determine 3D world coordinates from 2D image coordinates. Because a 2D image lacks depth information, the physical coordinates may reside anywhere on a specific line in 3D world coordinates. In order to determine where on the line the point resides, a hit test may be carried out to determine the point on the line that intersects the 3D mesh representing an object, such as a face. In some embodiments, Apple SceneKit's hitTest( ) method may be used. A scale factor for a three-dimensional image, SF_{3D}, may be determined from the approximate depth of the pupils, Zp, which is found from the 3D position of the pupils on the face mesh determined by the hit test, and the focal length of the camera, f, which is known, using the following equation:

$$S {F}_{3 D} = \frac{{Z}_{P}}{f} \qquad Eq . 8$$

At block 316B, the process may perform fine object localization. In some embodiments, the fine object localization is performed in a similar manner as described above with respect to block 316A. For example, fine object localization may be performed using one or more of the methods described in U.S. Patent Application Publication No. 2021/0393121 and result in an image as shown in FIG. 4F.

At block 318B, the coordinates for one or more landmarks may be calculated. For example, the locations of the landmarks in the image, such as those shown in FIGS. 4A-4F and/or FIGS. 5A-5E having locations in a 3D face mesh, may be converted to physical coordinates using the scaling factor SI3D, as will be understood by one of ordinary skill in the art.

At block 320B, one or more measurements may be calculated from the landmark coordinates determined at block 318B. The one or more measurements may be calculated in the same manner as described above with respect to block 320A. For example, Equations 3-7 may be used to calculate the various segment heights.

The measurements determined or calculated at block 320B may be stored locally or in a remote data storage as block 212 in FIG. 2. For example, in some embodiments, the measurement values may be saved in a data store 150 at or in communication with management server 130 such that the measurement values are associated with a profile of the user.

The present disclosure can be embodied in the form of methods and apparatuses for practicing those methods. The present disclosure can also be embodied in the form of program code embodied in tangible media, such as secure digital (“SD”) cards, USB flash drives, diskettes, CD-ROMs, DVD-ROMs, Blu-ray disks, hard drives, or any other non-transitory machine-readable storage medium, wherein, when the program code is loaded into and executed by a machine, such as a computer or processor, the machine or processor becomes an apparatus for practicing the disclosure. The present disclosure can also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer or processor, the machine becomes an apparatus for practicing the disclosure. When implemented on a general-purpose processor, the program code segments combine with the processor to provide a unique device that operates analogously to specific logic circuits.

The above-described embodiments are merely possible examples of implementations, and merely set forth a clear understanding of the principles of the disclosure. Many variations and modifications may be made to the above-described embodiments and examples of the disclosure without departing substantially from the spirit and principles of the disclosure. All such modifications and variations are intended to be included herein within the scope of this disclosure and the present disclosure and protected by the following claims.

While this specification contains many specifics, these should not be construed as limitations on the scope of any disclosure or of what may be claimed, but rather as descriptions of features that may be specific to particular embodiments or examples of particular disclosures. Certain features that are described in this specification in the context of separate embodiments or examples may also be implemented in combination in a single embodiment or example. Conversely, various features that are described in the context of a single embodiment may also be implemented in multiple embodiments or examples separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

While various embodiments have been described, it is to be understood that the embodiments described are illustrative only and that the scope of the subject matter is to be accorded a full range of equivalents, many variations and modifications naturally occurring to those of skill in the art from a perusal hereof.

1. A method, comprising:
obtaining an image, the image including a first object and a second object;
localizing, using at least one processor, the second object in the image;
determining, using the at least one processor, a scaling factor based on at least one of the first object in the image and the second object in the image;
converting, using the at least one processor, coordinates of a first landmark associated with the first object in the image and a second landmark associated with the second object in the image from a first unit of measure to a second unit of measure based on the scaling factor;
calculating, using the at least one processor, at least one measurement between the first landmark associated with the first object in the image and the second landmark associated with the second object in the image; and
storing the at least one measurement in a non-transitory machine readable storage medium that is disposed in communication with the at least one processor.

2. The method of claim 1, wherein the image includes two-dimensional image data.

3. The method of claim 2, wherein determining the scaling factor includes:
determining, using the at least one processor, a first dimension of the first object in the image, the first dimension of the first object in the image being in a first unit of measure; and
dividing the first dimension of the first object by a value in the second unit of measure.

4. The method of claim 3, further comprising obtaining the value in the second unit of measure from a model of the first object.

5. The method of claim 3, wherein the value of the second unit of measure is a predetermined constant value.

6. The method of claim 3, wherein the first object includes frames for spectacles, and wherein the value corresponds to a height of a lens for the frames.

7. The method of claim 6, wherein determining the first dimension includes determining a distance between the first landmark associated with the frames for spectacles in the image and a third landmark associated with the frames for spectacles in the image.

8. The method of claim 1, wherein determining the scaling factor includes:
determining a distance from a camera to the second object; and
dividing the distance from the camera to the second object by a focal length of the camera.

9. The method of claim 8, wherein the second object includes a pupil.

10. The method of claim 1, wherein the image is obtained from a camera in communication with the at least one processor, and wherein the method includes segmenting, using the at least one processor, the first object in the image.

11. The method of claim 1, wherein the image is a composite image based on a first image obtained from a camera and a model of the first object superimposed on the first image.

12. A non-transitory machine readable storage medium having machine-executable instructions stored therein, wherein, when executed by one or more processors, the one or more processors perform a method, the method comprising:
obtaining an image, the image including a first object and a second object;
localizing, using the at least one processor, the second object in the image;
determining, using the at least one processor, a scaling factor based on at least one of the first object in the image and the second object in the image;
converting, using the at least one processor, coordinates of a first landmark associated with the first object in the image and a second landmark associated with the second object in the image from a first unit of measure to a second unit of measure based on the scaling factor;
calculating, using the at least one processor, at least one measurement between the first landmark associated with the first object in the image and the second landmark associated with the second object in the image; and
storing the at least one measurement in a non-transitory machine readable storage medium that is disposed in communication with the at least one processor.

13. The non-transitory machine readable storage medium of claim 12, wherein the image includes two-dimensional image data.

14. The non-transitory machine readable storage medium of claim 13, wherein determining the scaling factor includes:
determining, using the at least one processor, a first dimension of the first object in the image, the first dimension of the first object in the image being in a first unit of measure; and
dividing the first dimension of the first object by a value in the second unit of measure.

15. The non-transitory machine readable storage medium of claim 14, wherein the method includes obtaining the value in the second unit of measure from a model of the second object.

16. The non-transitory machine readable storage medium of claim 14, wherein the value of the second unit of measure is a predetermined constant value.

17. The non-transitory machine readable storage medium of claim 14, wherein the first object includes frames for spectacles, and wherein the value corresponds to a height of a lens for the frames.

18. The non-transitory machine readable storage medium of claim 17, wherein determining the first dimension includes determining a distance between the first landmark associated with the frames for spectacles in the image and a third landmark associated with the frames for spectacles in the image.

19. The non-transitory machine readable storage medium of claim 12, wherein determining the scaling factor includes:
determining, using the at least one processor, a distance from a camera to the second object; and
dividing the distance from the camera to the second object by a focal length of the camera.

20. (canceled)

21. (canceled)

22. (canceled)

23. A computing device, comprising:
a non-transitory machine readable storage medium; and
at least one processor in communication with the non-transitory machine readable storage medium, the at least one processor configured to:
obtain an image including a first object and a second object;
localize the second object in the image;
determine a scaling factor based on at least one of the first object in the image and the second object in the image;
convert coordinates of a first landmark associated with the first object in the image and a second landmark associated with the second object in the image from a first unit of measure to a second unit of measure based on the scaling factor;
calculate at least one measurement between the first landmark associated with the first object in the image and the second landmark associated with the second object in the image; and
store the at least one measurement in the non-transitory machine readable storage medium.

24. The computing device of claim 23, wherein the image includes two-dimensional image data.

25. The computing device of claim 24, wherein the at least one processor is configured to:
determine a first dimension of the first object in the image, the first dimension of the first object in the image being in a first unit of measure; and
divide the first dimension of the first object by a value in the second unit of measure to determine the scaling factor.

26. The computing device of claim 25, wherein the at least one processor is configured to obtain the value in the second unit of measure from a model of the first object.

27. (canceled)

28. (canceled)

29. (canceled)

30. (canceled)

31. The computing device of claim 25, wherein the at least one processor is configured to determine a distance between the first landmark associated with the frames for spectacles in the image and a third landmark associated with the frames for spectacles in the image to determine the first dimension.

32. The computing device of claim 23, wherein the at least one processor is configured to:
determine a distance from a camera to the second object; and
divide the distance from the camera to the second object by a focal length of the camera to determine the scaling factor.

33. (canceled)

34. (canceled)

35. (canceled)
